(19) United States
(12) Plant Patent Application Publication
(10) Pub. No.: US 2010/0024085 P1
(43) Pub. Date: Jan. 28, 2010

(54) Mandevilla LINDL

(50) ;

(75) Inventor: Norio Sonoda, Kagoshima (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Norio Sonoda, Aira-gun (JP)

(21) Appl. No.: 12/458,644

(22) Filed: Jul. 17, 2009

(30) Foreign Application Priority Data
Jul. 17, 2008 (JP) 22770

(51) Int. Cl. A01H 5/00

(52) U.S. Cl. PLT/232

(57) ABSTRACT

A new and distinct Mandevilla plant named “Tsuruhanbestpinki1” of upright and vining plant habit, relatively small glossy foliage, freely branching habit, freely flowering habit, and being in full flower around Mother's Day as illustrated.

### NAME OF PLANT

Mandevilla.Lindl.

### NAME OF VARIETY

“Tsuruhanbestpinki1”

This application claims priority of Japanese application for variety registration no. 22770, filed on Jul. 17, 2008. All content of the application is incorporated herein by reference.

### BACKGROUND OF THE INVENTION

In May, 2003, to obtain this variety, a pink-based nameless variety of a culture line possessed by Applicant was crossbred with mixed pollens of nameless varieties of a culture line possessed by Applicant in a farm owned by Applicant (Yusui-cho, Aira-gun, Kagoshima Pref.).

In February, 2004, a resultant seed was inoculated.

In April, 2005, from the flowering plants, individual roots were selected and started to be grown by cutting.

In June, 2006, the culture was completed after ensuring uniformity and stability.

### SUMMARY OF THE INVENTION

1. Upright and vining plant habit.
2. Relatively small glossy foliage.
3. Freely branching habit.
4. Freely flowering habit.
5. Being in full flower around Mother's Day.

### BRIEF DESCRIPTION OF THE PHOTOGRAPHS

The accompanying colored photographs illustrate the overall appearance of the new cultivar, showing the colors as true as it is reasonably possible to obtain in colored reproductions of this type. Colors in the photographs may differ slightly from the color values cited in the detailed botanical description which accurately describe the actual colors of the new Mandevilla.

The photograph at the top of the sheet comprises a front perspective view of a typical flowering plant of “Tsuruhanbestpinki1” grown in a greenhouse.

The photograph at the bottom of the sheet is a close-up view of typical flowers of “Tsuruhanbestpinki1”.

FIG. 1 is a panoramic view of the cultivation area.

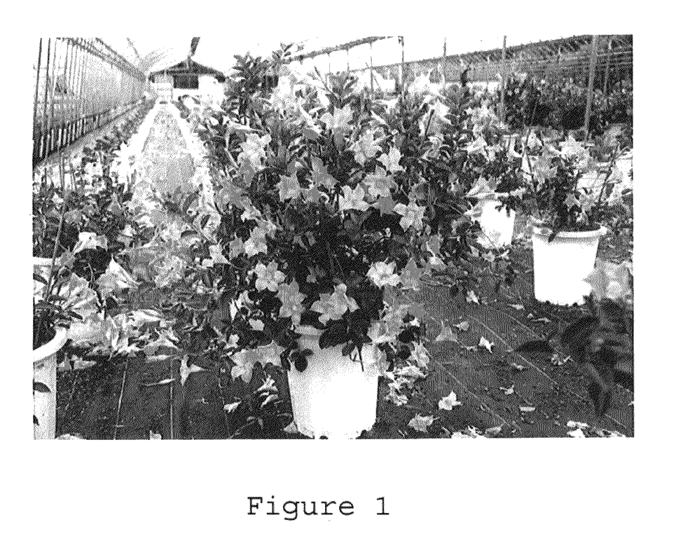

FIG. 2 is a close-up of the flower.

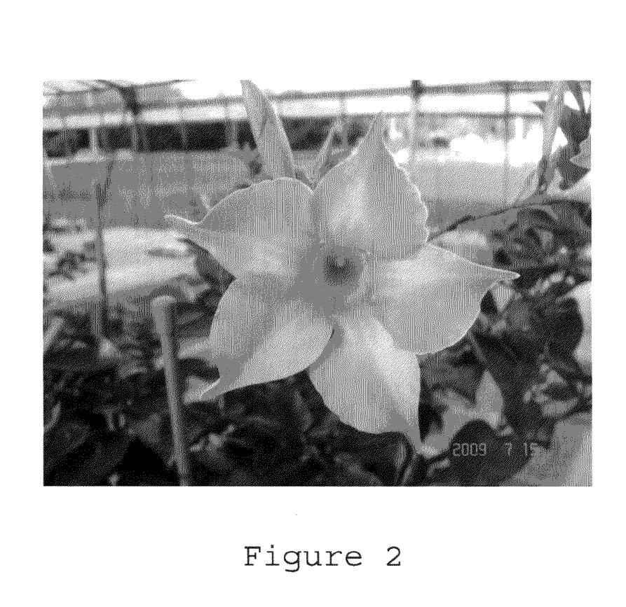

FIG. 3 is a diagram of the plant body.

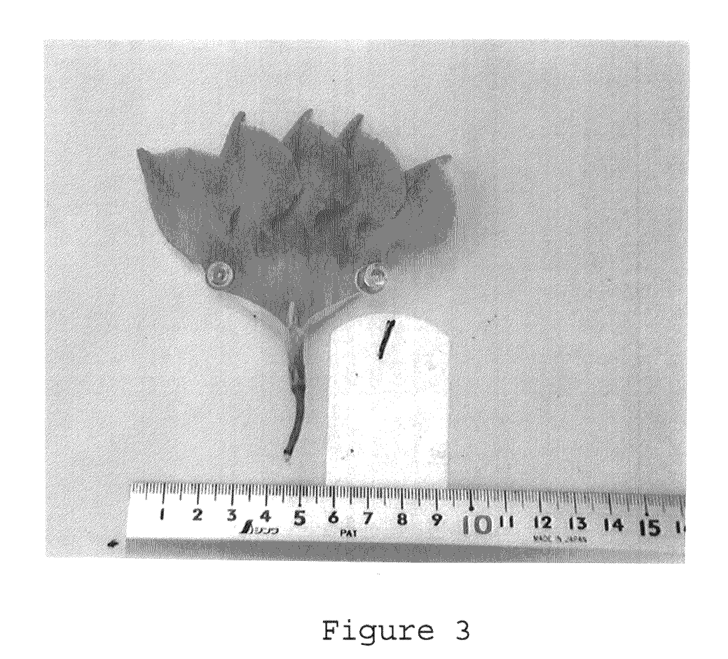

FIG. 4 is a plant variety for comparison.

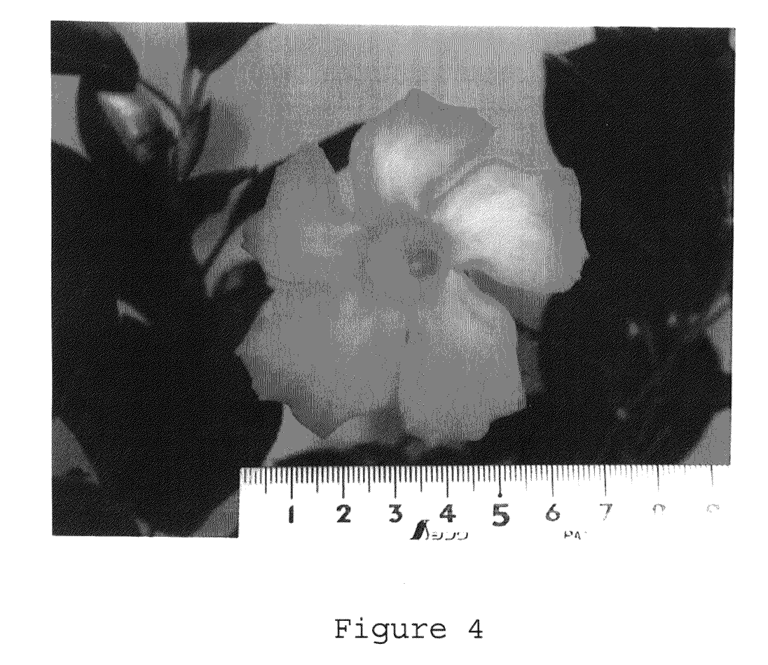

FIG. 5 is a plant variety for comparison.

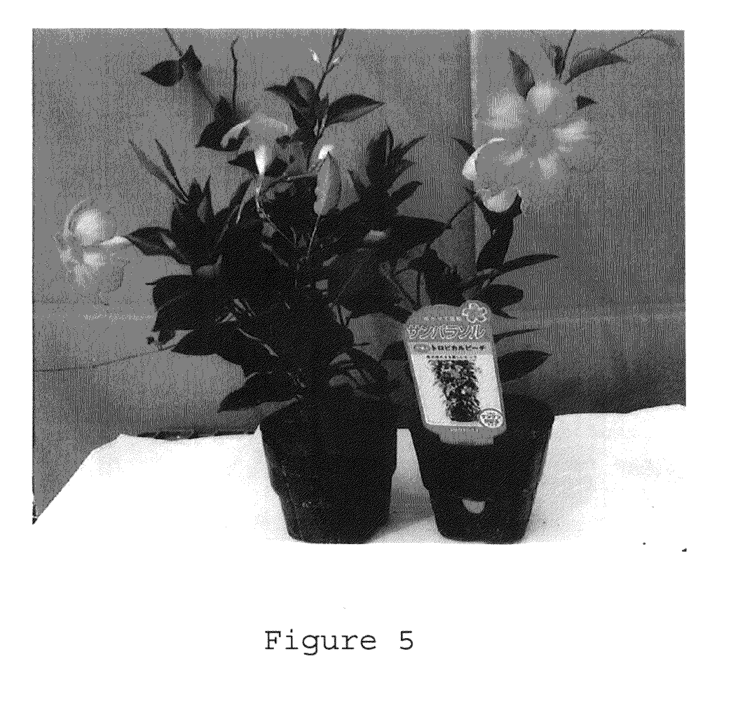

### DETAILED BOTANICAL DESCRIPTION

In the following description, color references are made to Japan Horticulture Standard Color Chart, edited as the 2nd print, resale on Sep. 10, 1997, except where general terms of ordinary dictionary significance are used. Plants used for the aforementioned photographs and following description were grown under conditions which closely approximate commercial production conditions during the early summer in a polyethylene covered greenhouse in Yusui-cho, Aira-gun, Kagoshima Pref. in Japan. During the production of the plants, day temperatures averaged 26° C., and night temperatures averaged 14° C.
Propagation:
   Type.—By vegetative cuttings.
   Time to initiate roots.—About three weeks at 23° C. to 26° C.
   Time to produce a rooted young plant.—About 40 days at 23° C. to 26° C.
   Root description.—Fibrous, fleshy, white in color.
   Rooting habit.—Freely branching.
Plant description:
   Form.—Perennial — Upright and vining plant habit. Compact growth habit.
   Plant height.—About 54 cm.
Lateral branch description:
   Length.—About 54 cm.
   Diameter.—About 3 mm.
   Internode length.—About 2 cm.
   Strength.—Strong.
   Texture.—Smooth, glabrous.
   Color, young.—Close to Color Chart No. 3307.
   Mature.—Close to Color Chart No. 1309.
Foliage description:
   Arrangement.—Opposite, simple.
   Length.—About 6.2 cm.
   Width.—About 3.5 cm.
   Shape.—Long elliptic.
   Apex.—Cuspidate.
   Base.—Circular.
   Margin.—Entire green.
   Texture, upper and lower surfaces.—Smooth, glabrous.
   Luster, upper and lower surfaces.—Glossy.
   Venation pattern.—Pinnate, reticulate.
Color:
   Developing foliage, upper surface.—Close to Color Chart No. 3508.
   Developing foliage, lower surface.—Close to Color Chart No. 3508.
   Fully expanded foliage, upper surface.—Close to Color Chart No. 3509, Venation, Close to Color Chart No. 3507.
   Lower surface.—Close to Color Chart No. 3508, Venation, Close to Color Chart No. 3508.
   Petiole length.—About 1.3 cm.
   Petiole diameter.—About 0.1 cm.
   Petiole texture, upper and lower surfaces.—Sparsely pubescent.
   Petiole color, upper and lower surfaces.—Close to Color Chart No. 3507.
Flower description:
   Flower type and habit.—Single salver form flowers; flowers racemose; flowers face mostly outwardly, freely flowering habit, about five flowers per inflorescence.
   Natural flowering season.—Long flowering period; Fully flowering from late spring to early summer in Japan; Rarely flowering in midsummer, and then be in a flowering period again in late autumn.
   Flower longevity on the plant.—About ten to fourteen days. Flowers not persistent.
   Fragrance.—Not detected.
   Inflorescence length.—9.5 cm.
   Inflorescence diameter.—8.0 cm.
Flowers:
   Appearance.—Flared trumpet, corolla fused, five-parted; petals slightly imbricate; flowers roughly star-shaped.
   Diameter.—8.0 cm.
   Depth (length).—9.5 cm.
   Throat diameter.—About 1.8 cm.
   Tube length.—About 5.5 cm.
   Tube diameter, mid-section.—2.0 cm.
   Tube diameter, base.—About 3 mm.
Flower buds:
   Height.—About 7.5 cm.
   Diameter.—About 1.3 cm.
   Shape.—Lenticular.
   Color.—Close to Color Chart No. 9704.
Corolla:
   Arrangement/appearance.—Single whorl of five petals, fused into flared trumpet; petals slightly imbricate.
   Petal length.—About 3.5 cm.
   Petal width.—About 2.5 cm.
   Petal shape.—Spatulate.
   Petal apex.—Acuminate.
   Petal margin.—Entire.
   Petal texture, upper and lower surfaces.—Smooth, glabrous; velvety.
   Color.—Petal, when opening and fully opened, upper surface: Close to Color Chart No. 9504; random sectors, Close to Color Chart No. 9504. Lower surface: Close to Color Chart No. 0103; random sectors, Close to Color Chart No. 0103.
Sepals:
   Arrangement/appearance.—Five per flower in a single whorl; fused.
   Length.—About 10 mm.
   Width.—About 4 mm.
   Shape.—Lanceolate.
   Apex.—Acute.
   Margin.—Entire.
   Texture, upper and lower surfaces.—Smooth, glabrous.
   Color, immature, upper and lower surfaces.—Close to Color Chart No. 3504; towards the apex, close to Color Chart No. 3504.
   Color, mature, upper and lower surfaces.—Close to Color Chart No. 3504; towards the apex, close to Color Chart No. 3504.
Peduncles:
   Length.—About 8.0 cm.
   Diameter.—About 3.5 mm.
   Texture.—Smooth, glabrous.
   Strength.—Flexible, but strong.
   Color.—Close to Color Chart No. 3507.
Pedicels:
   Length.—About 2.2 cm.
   Diameter.—3mm.
   Texture.—Smooth, glabrous, flexible, but strong.
   Color.—Close to Color Chart No. 3507.
Reproductive organs:
   Stamens.—Quantity/arrangement: Typically five; filament fused to corolla; anthers, connivent.
   Anther shape.—Elliptic.
   Anther size.—About 8 mm by 1 mm.
   Anther color.—Close to Color Chart No. 2505.
   Pollen amount.—Scarce.
   Pollen color.—Close to Color Chart No. 2506.
   Pistils.—Quantity: Typically one.
   Pistil length.—About 2.2 cm.
   Stigma shape.—Conical.
   Stigma color.—Close to Color Chart No.3506.
   Ovary color.—Close to Color Chart No. 3506.
Seed/fruit: Seed and fruit production has not been observed.
Disease/pest resistance: Plants of the new Mandevilla have not been noted to be resistant to pathogens and pests common to Mandevilla.
Temperature tolerance: Plants of the new Mandevilla have been observed to tolerate temperatures from about 5° C. to about 37° C.

What is claimed is:

1. A new and distinct Mandevilla plant named “Tsuruhanbestpinki1” as illustrated and described.
